(19) United States
(12) Patent Application Publication
(10) Pub. No.: US 2006/0178652 A1
(43) Pub. Date: Aug. 10, 2006

(54) Incontinence pad and apparel formed therewith

(76) Inventor: Robert A. Miller III, New Ringgold, PA (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(21) Appl. No.: 11/054,643

(22) Filed: Feb. 9, 2005

(51) Int. Cl. A61F 13/15

(52) U.S. Cl. 604/385.101

(57) ABSTRACT

An absorbent, multi-layer, moisture management pad is provided for wear in the crotch area of apparel. The pad includes multiple permeable layers. At least one of the layers has alternating ridges and valleys for directing moisture in one direction across its surface area. At least one of the layers has alternating ridges and valleys for directing moisture in a second direction across its surface area, wherein the surface areas and volumes of the respective layer are maximized.

### FIELD OF THE INVENTION

The present invention relates to the field of textile production, and, more particularly to a multi-layer, absorbent pad, and to garments incorporating the multi-layer, absorbent pad.

### BACKGROUND OF THE INVENTION

Incontinence, or the inability to control excretory functions, is a common condition, particularly for infants and the elderly. Diapers and absorbent briefs have been developed over the years for wear by incontinent persons. These diaper and brief constructions typically include liquid a permeable inner layer, an intermediate liquid absorbent layer, and an outer liquid impervious layer. The inner layer, which is generally in contact with the wearer's skin, aids in moving the liquid away from the wearer's skin. The intermediate absorbent layer contains the liquid, and the bottom impervious layer prevents liquid from penetrating the diaper or brief and wetting the wearer's outer apparel.

Various structures for absorbent briefs and pads have been developed for quickly absorbing rapid, large volumes of liquid, such as urine. Some of these structures have employed layers of perforated absorbent material. Others have provided channels or canals for containing the liquid and/or moving the liquid in a certain direction. In most cases, however, and quite ineffectively, only a fraction of the absorbent surface area and volume of the brief or pad are utilized.

### BRIEF DESCRIPTION OF THE DRAWINGS

FIG. 1 is a front view of the panty construction of the present invention;

FIG. 2 is a rear view of the panty construction of FIG. 1;

FIG. 3 is a top view of the crotch section of the panty construction of FIG. 1;

FIG. 4 is a partial sectional side view of the panty construction of FIG. 1;

FIG. 5 is an exploded cross-sectional view of one embodiment of the multi-layer, liquid absorbent pad of the present invention;

FIG. 6 is an exploded cross-sectional view of an alternative embodiment of the multi-layer, liquid absorbent pad of the present invention;

FIG. 7A is a top view of a liquid permeable, absorbent layer having a plurality of apertures;

FIG. 7B is a bottom view of the liquid permeable, absorbent layer of FIG. 5A, having a plurality of dimples;

FIG. 8A is a top view of a liquid permeable, absorbent layer having alternating ridges and valleys perpendicular to the major axis of the crotch section of the panty construction;

FIG. 8B is a top view of the liquid permeable, absorbent layer of FIG. 6A illustrating the manner in which the layer distributes moisture;

FIG. 9A is a top view of a liquid permeable, absorbent layer having diagonally oriented ridges and valleys across the surface of the layer;

FIG. 9B is a top view of the liquid permeable, absorbent layer of FIG. 7A illustrating the manner in which the layer distributes moisture;

FIG. 10A is a top view of a liquid permeable, absorbent layer having alternating ridges and valleys cross the width of the crotch section of the panty construction; and

FIG. 10B is a top view of the liquid permeable, absorbent layer of FIG. 8A illustrating the manner in which the layer distributes moisture.

### DETAILED DESCRIPTION OF THE PREFERRED EMBODIMENTS

Referring to the Figures in general, the present invention is directed to a multi-layer, absorbent moisture management pad and to a panty construction having a multi-layer, absorbent moisture management pad incorporated therein.

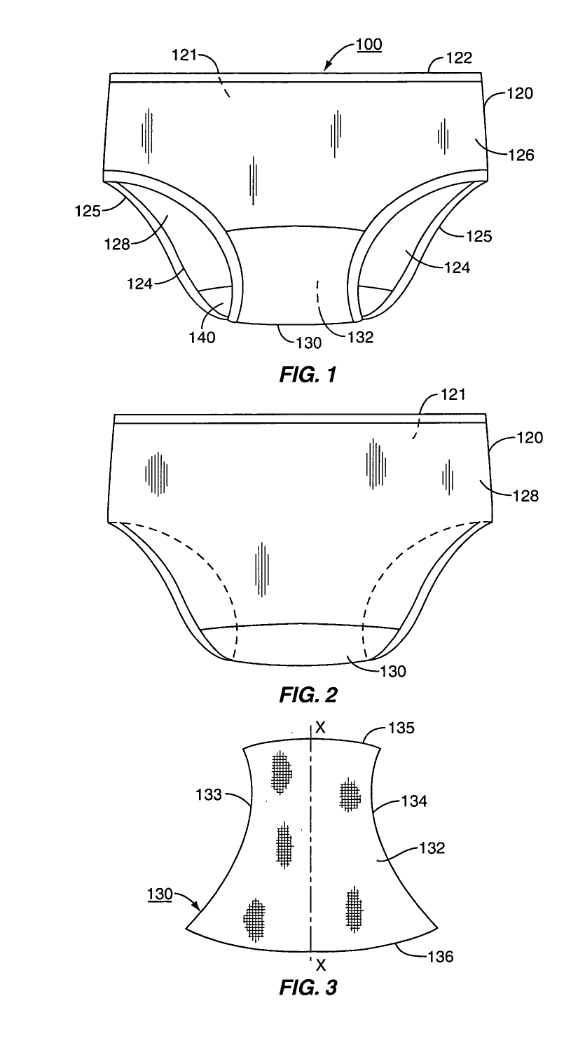

FIGS. 1 through 3 illustrate a panty construction having a moisture management, multi-layer, absorbent pad incorporated into the crotch of the panty. Shown generally as 100, the panty construction comprises a body portion 120 and a multi-layer, absorbent pad 140.

The body portion 120 is conventionally constructed from one or more panels, or pieces, of fabric. The body portion 120 may be formed of 100 percent cotton yarns; polyester, nylon, rayon, and combinations or intimate blends may also be used in constructing the body 120. The body portion 120 may be knitted or woven, depending upon the characteristics desired in the final constructed garment. When knitted, the body may be circular or warp knitted.

The body portion 120 shown in FIGS. 2 and 3 comprises a waist opening 121 at the top of the body, a pair of leg openings 124, a front section 126, a back section 128, and a crotch section 130. While each of these components of the body portion 120 would typically be formed of the same material, the invention is not limited thereto. Rather, each portion of the body could be formed from a different type of material; e.g., the front section 126 could be nylon and the back section 128 could be cotton, etc. As is typical in panty constructions, a waistband 122 including an elastomeric material may be sewn, or otherwise attached around the periphery of the waist opening 121. Similarly, elastomeric trim 125 may be affixed around the leg openings 124 to secure them snugly against the legs of the wearer.

The crotch section 130 may be formed from the same material, i.e., cotton, as the other components of the body portion 120. The crotch section 130 joins the front section 126 and the back section 128 and defines a major axis between the front 126 and back 128. The major axis is denoted as Line X-X in FIG. 3. As shown in FIGS. 1, 3 and 4, the crotch section 130 has an inner surface 132, opposed side edges 133, 134 adjacent the leg openings 124, and opposed front 135 and back 136 edges adjacent the front 126 and back 128 sections of the body 120. As is conventional in underwear, and panty, constructions, the crotch section 130 is stitched or welded around its periphery to front section 126 and back section 128 along the front and back edges 135, 136 of the crotch section. Similarly, the opposed side edges 133, 134 are stitched or welded at the leg openings 124.

Referring first to 5 and 6, two embodiments 142, 144 of the present invention, which will be described in greater detail below, are illustrated. These embodiments are exemplary of numerous possible embodiments to achieve the multi-layer, absorbent pad of the present invention. In each of the embodiments, the multi-layer, absorbent pad is constructed to move liquid, or moisture, away from the wearer skin as rapidly as possible, while maximizing the surface areas and volume of the pad by directing the liquid in sequential and multiple directions throughout the multi-layer construction. As will be appreciated, the multi-layer pad 140 may be formed in a number of different configurations, such as 142, 144, with the arrangement of layers varying from one construction, or embodiment, to another, depending on the particular application or end use of the pad 140; however, all are within the scope of the present invention.

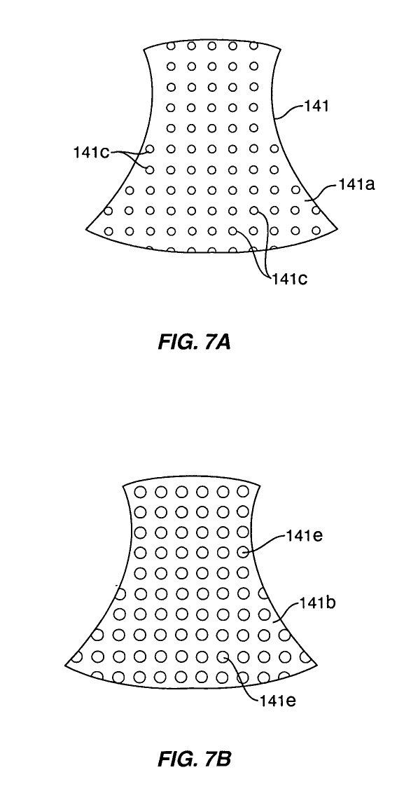

Turning now to FIGS. 7 through 10, several exemplary layers that may be used to form a multi-layer, absorbent pad 140 are shown in top plan view. By “top,” we mean closest to the wearer. While each of the layers is liquid-permeable, at least some of the layers are formed of liquid-permeable, absorbent, non-woven material. As used herein, “non-woven” material refers to an assembly of textile fibers that are held together by mechanical interlocking in a random web or mat, by fusing the fibers, or by bonding the fibers with a cementing medium. The manner of forming thin, non-woven fabric is well known in the art and, and non-woven fabric layers may be formed with various surface features. One source of thin, non-woven fabric layers is Palmer Group, Inc. of Charlotte, N.C. Suitable materials for the non-woven structure comprise polyester, polypropylene, nylon, cotton, and blends thereof, however, the present invention is not limited thereto.

While the following description of the invention illustrates several different non-woven single-layer constructions, those skilled in the art will appreciate that there are countless variations in the way the layers are formed to direct and disburse liquid that may suitably provide the desired results when combined in a multi-layer, absorbent pad. One application of the multi-layer, absorbent pad 140 of the present invention is for incorporation into a non-disposable (reusable) panty or brief for incontinent adults. As such, certain embodiments of the pad may comprise one or more permeable, absorbent layers that are chemically treated, or finished, with anti-microbial and/or odor-inhibiting treatments. For example, one or more of the layers may be treated with a finish such as NIMBUS™ (Novel Intrinsically Microbiocidal Utility Substance), available from Quick-Med Technologies, Inc. of Gainesville, Fla. NIMBUS™ is an anti-microbial, super-absorbent finish for, among others materials, synthetics that are otherwise hydrophobic if untreated. Alternatively, other chemical finishes, such as THERMOSENSE, available from Thermosense Incorporated of Vancouver, BC, is also an odor-inhibiting, anti-bacterial/microbial, absorbent material that may impart similar physical and chemical properties to one or more of the non-woven layers forming the pad 140.

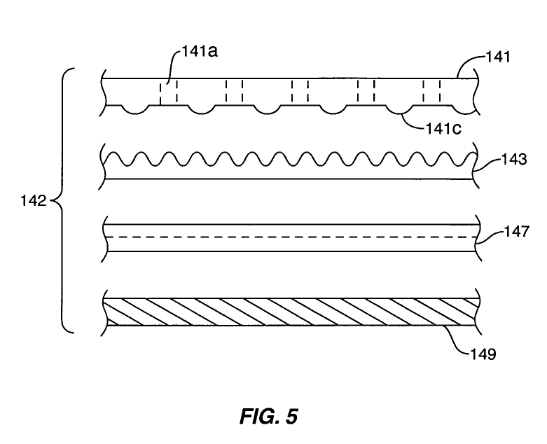

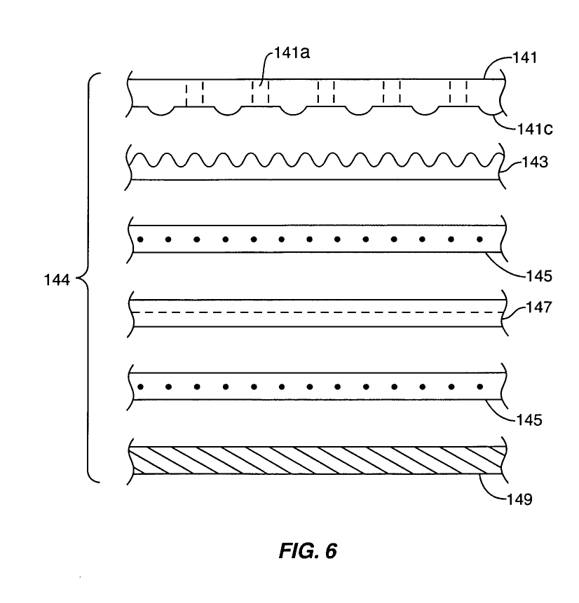

Referring to FIGS. 7A and 7B, on exemplary layer 141 comprises a thin non-woven sheet having a pattern of apertures 141c formed therethrough the layer 141 for the rapid passage of moisture downwardly from the top of the layer 141a. As will be illustrated in the embodiments of FIGS. 5 and 6 below, this apertured layer 141 may comprise the first, or top, layer of the multi-layer pad and that is in contact with the wearer's skin. As best shown in the bottom plan view of FIG. 7B, and in FIGS. 5 and 6, a pattern of downwardly projecting dimples 141e are formed on the bottom side 141b of this layer 141. The dimples 141e maintain a relative separation between the top layer 141 and an underlying adjacent absorbent layer. This separate facilitates a more rapid movement of liquid through the apertures 141c and into the adjacent lower layer, while minimizing the time that liquid is in contact with the wearer's skin. As those skilled in the art will appreciate, the patterns, sizes, and shapes of the apertures 141c and dimples 141e are not important, so long as they are formed for anticipated liquid discharge by the wearer.

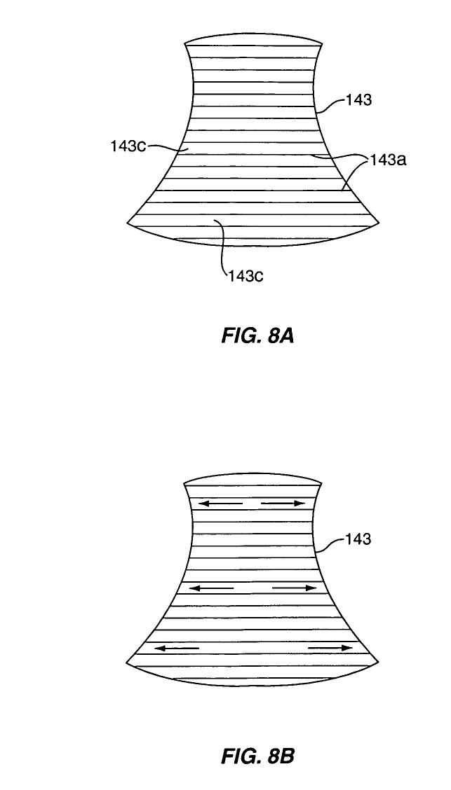

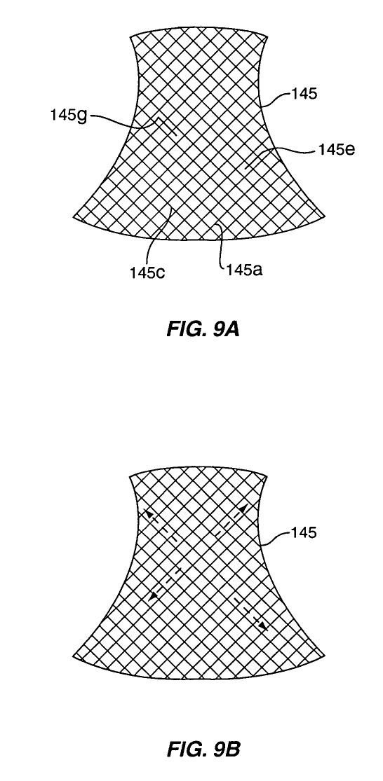

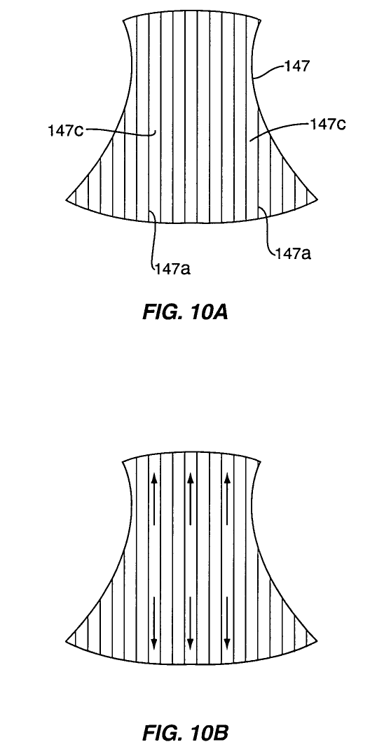

Turning to FIGS. 8A and 8B, another exemplary layer 143 is shown. This exemplary layer 143 comprises a plurality of alternating ridges 143a and valleys 143c, or channels, that are substantially parallel. As best shown by the arrows in FIG. 8B, the alternating ridges 143a and valleys 143c extend substantially perpendicular to the major axis of the crotch and layer 143 so that liquid or moisture is directed across the width of the layer 143. In the exemplary layer constructions of FIGS. 8, as well as FIGS. 9 and 10, the ridges are preferably at least about 1/32 inches in height, and the valleys, or channels, are preferably at least about 1/32 inches wide.

Turning to FIGS. 9A and 9B, a third exemplary layer 145 is illustrated. Layer 145 comprises a crisscrossing or diagonal pattern of alternating ridges 145a, 145c and valleys 145e, 145g. These ridges 145a, 145c and valleys 145e, 145g direct liquid diagonally across substantially the entire surface of the layer 145, as best shown in FIG. 6B.

Lastly, a fourth exemplary layer 147 is illustrated in FIGS. 10A and 10B. This exemplary layer 147 comprises a plurality of substantially parallel alternating ridges 147a and valleys 147c, or channels. As best shown by the arrows in FIG. 10B, the alternating ridges 147a and valleys 147c extend substantially parallel to the major axis of the crotch and layer 147 so that liquid or moisture is directed along the entire length of the layer 147.

Turning again to FIG. 5, a cross-section of one exemplary construction of the multi-layer, absorbent pad 142 is shown. The pad 142 may comprise a first top layer 141 as described above, wherein the top layer 141 has a plurality of apertures 141a for moving moisture away from a wearer's skin, and a plurality of spaced apart, downwardly projecting dimples 141c for maintaining relative separation between the top layer 141 and the underlying adjacent absorbent layer 143. Liquid passing through the top layer 141 is both absorbed by layer 141 and passes quickly passed downwardly through the apertures 141c where it contacts absorbent layer 143. As described above, alternating ridges 143a and valleys 143c direct moisture in a first direction across the width of the entire layer 143. Liquid passing through the absorbent non-woven layer 143 contacts the adjacent underlying layer 147. As described above, the alternating ridges 147a and valleys 147c both absorb and direct moisture in a second direction along the length and area of the layer 147. The intermediate absorbent layers may be stacked so that adjacent layers are not of the same type; i.e., two layers 147 would not be stacked adjacent one another. A bottom layer 149 may be provided that is substantially impervious to liquid. The bottom layer 149 serves to trap liquid at the bottom of the pad 142 so that it does not seep through or leak into the wearer's outer apparel. A suitable bottom layer 149 may be formed as a synthetic film, a chemically treated fabric, or other material that can be constructed or treated to make the layer substantially impervious to liquid.

Turning again to FIG. 6, another exemplary cross-section of the multi-layer, absorbent pad 144 is shown. This pad 144 is merely exemplary of one of the many pad constructions that are possible when constructed with the various non-woven layers described above. Again, desirably the construction is formed with adjacent layers that direct liquid in different directions across and within the pad 144. Again, the pad 144 desirably comprises a first top layer 141 as described above, wherein the a top layer 141 has a plurality of apertures 141a for removing moisture away from a wearer's skin, and a plurality of spaced apart, downwardly projecting dimples 141c for maintaining relative separation between the top layer 141 and the underlying adjacent absorbent layer 143. Liquid passing through the top layer 141 is both absorbed by layer 141 and quickly passes downwardly through the apertures 141 where it contacts absorbent layer 143. As described above, alternating ridges 143a and valleys 143c direct moisture in a first direction across the width of the entire layer 143, while absorbing some volume of the liquid. Liquid passing through the layer 143 next contacts adjacent layer 145. As described above, layer 145 comprises a crisscrossing, diagonal pattern of alternating ridges 145a, 146c and valleys 145e, 145g. These ridges 145a, 145c and valleys 145e, 145g direct liquid diagonally across substantially the entire surface of the layer 145, and may also absorb some volume of the liquid. Liquid passing through the absorbent non-woven layer 145 next contacts the adjacent underlying layer 147. The alternating ridges 147a and valleys 147c both absorb and direct moisture along the length and area of the layer 147. The liquid next passes into a second layer 145, where it is dispersed and absorbed within the crisscrossing diagonal pattern of ridges and valleys. Lastly, a bottom layer 149 is provided that is substantially impervious to liquid.

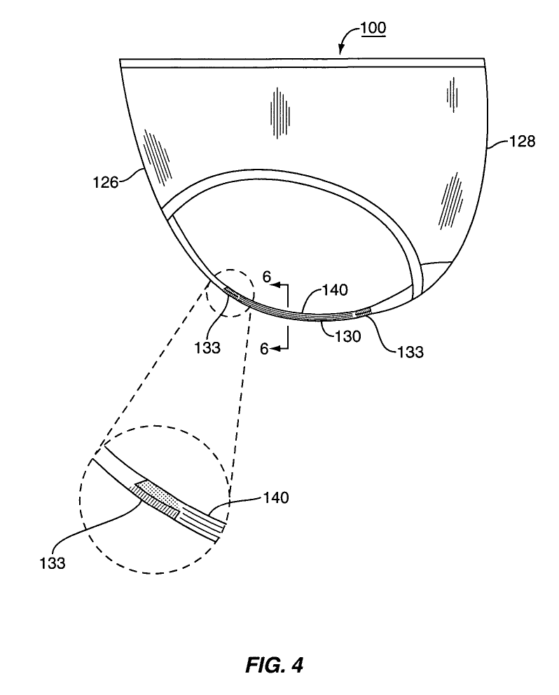

When placed inside the crotch section 130, the moisture management pad 140, comprising multiple layers such as the constructions shown as 142, 144, is oriented so that the major axis of the pad 140 corresponds to the major axis of the crotch section 130. In one embodiment, the layers of the pad 140 may first be stitched or welded around some portions, or all, of their opposed side and opposed end edges to hold the pad together. Stitching or welding around the side and end edges also provides a barrier to prevent liquid from seeping out the sides of ends of the pad 140, 142, 144. The completed pad may then be stitched or welded inside the crotch section 130. Alternatively, the layers may be stacked within the crotch section and so that one stitching or welding operation will complete the pad construction and attach the pad 140 to the crotch section 130. Where a removable pad is desired, the pad and crotch section may be provided with a removable fastening material such as the hook and loop material 133 illustrated schematically in FIG. 4. Other fasteners such as snaps, buttons, tapes, slings, harnesses, and pockets, etc. may also be employed for this purpose.

Although the present invention has been described with preferred embodiments, it is to be understood that modifications and variations may be utilized without departing from the spirit and scope of the invention, as those skilled in the art will readily understand. Such modifications and variations are considered to be within the purview and scope of the appended claims and their equivalents.

We claim:

1. An absorbent, multi-layer, moisture management pad for wear in the crotch area of apparel, comprising:
(a) a plurality of permeable layers, each having a surface area, opposed ends defining a major axis therebetween, and opposed sides defining a width therebetween;
(b) at least one of the plurality of permeable layers comprising alternating ridges and valleys for directing moisture in a first direction across its surface area; and
(c) at least one of the plurality of absorbent, permeable layers comprising alternating ridges and valleys for directing moisture in a second direction across its surface area.

2. The moisture management pad of claim 1 wherein the permeable layers are selected from the group consisting of a layer having alternating ridges and valleys formed substantially parallel to a major axis, a layer having alternating ridges and valleys formed substantially perpendicular to the major axis, and a layer having alternating ridges and valleys formed substantially diagonal to the major axis.

3. The moisture management pad of claim 1 wherein the permeable layers are formed of non-woven material.

4. The moisture management pad of claim 3 wherein the non-woven material is selected from the group consisting of polyester, polypropylene, nylon, cotton, and blends thereof.

5. The moisture management pad of claim 1 further including a top layer comprising a plurality of apertures for removing moisture through the top layer away from a wearer's skin.

6. The moisture management pad of claim 5 wherein the top layer further comprises a bottom surface having a plurality of spaced apart, downwardly projecting dimples.

7. The moisture management pad of claim 1 further including a bottom layer that is substantially moisture impervious.

8. An absorbent, multi-layer, moisture management pad for wear in the crotch area of apparel, comprising:
(a) a permeable top layer comprising a plurality of apertures for removing moisture away from a wearer's skin;
(b) a plurality of intermediate permeable layers, each having a surface area, opposed ends defining a major axis therebetween, and opposed sides defining a width therebetween;
(c) at least one of the plurality of intermediate permeable layers comprising alternating ridges and valleys for directing moisture in a first direction across its surface area;
(d) at least one of the plurality of intermediate permeable layers comprising alternating ridges and valleys for directing moisture in a second direction across its surface area.; and
(e) a bottom layer that is substantially moisture impervious.

9. The moisture management pad of claim 1 wherein the permeable layers are selected from the group consisting of a layer having alternating ridges and valleys formed substantially parallel to a major axis, a layer having alternating ridges and valleys formed perpendicular to the major axis, and a layer having alternating ridges and valleys formed substantially diagonal to the major axis.

10. The moisture management pad of claim 1 wherein the permeable layers are formed of non-woven material.

11. The moisture management pad of claim 10 wherein the non-woven material is selected from the group consisting of polyester, polypropylene, nylon, cotton, and blends thereof.

12. The moisture management pad of claim 8 wherein the top layer further comprises a bottom surface having a plurality of spaced apart, downwardly projecting dimples.

13. A panty construction having improved moisture management, comprising:
(a) a body portion defining:
(i) a waist opening at its top and a pair of leg openings;
(ii) a front section and a back section;
(iii) a crotch section joining the front and back sections and defining a major axis therebetween, the crotch section having inner and outer surfaces, opposed side edges and opposed front and back edges;
(b) an absorbent, multi-layer, moisture management pad, including:
(i) a plurality of permeable layers, each having a surface area, opposed ens defining a major axis therebetween, and opposed sides defining a width therebetween;
(ii) at least one of the plurality of permeable layers comprising alternating ridges and valleys for directing moisture in a first direction across its surface area;
(iii) at least one of the plurality of permeable layers comprising alternating ridges and valleys for directing moisture in a second direction across its surface area; and
(c) wherein the moisture management pad overlies and is attached to the inner surface of the crotch section.

14. The panty construction of claim 13 wherein the permeable layers are selected from the group consisting of a layer having alternating ridges and valleys formed parallel to a major axis, a layer having alternating ridges and valleys formed substantially perpendicular to the major axis, and a layer having alternating ridges and valleys formed substantially diagonal to the major axis.

15. The panty construction of claim 13 wherein the permeable layers are formed of non-woven material.

16. The panty construction of claim 15 wherein the non-woven material is selected from the group consisting of polyester, polypropylene, nylon, cotton, and blends thereof.

17. The panty construction of claim 13 further including a top layer comprising a plurality of apertures for removing moisture away from a wearer's skin.

18. The panty construction of claim 17 wherein the top layer further comprises a bottom surface having a plurality of spaced apart, downwardly projecting dimples.

19. The panty construction of claim 13 further including a substantially moisture impervious bottom layer.

20. The panty construction of claim 13 wherein the moisture management pad is attached to the inner surface of the crotch section by stitching or welding around the periphery of at least some of the opposed side edges and opposed front and back edges of the crotch section.

21. The panty construction of claim 13 wherein the moisture management pad is releasably attached to the inner surface of the crotch section.

22. The panty construction of claim 21 wherein the moisture management pad is releasably attached by fasteners selected from the group consisting of snaps, buttons, tapes, hook and loop material, slings, harnesses, and pockets.

23. A panty construction having improved moisture management, comprising:
(a) a body portion defining:
(i) a waist opening at its top and a pair of leg openings;
(ii) a front section and a back section;
(iii) a crotch section joining the front and back sections and defining a major axis therebetween, the crotch section having inner and outer surfaces, opposed side edges and opposed front and back edges;
(b) an absorbent, multi-layer, moisture management pad, including:
(i) a permeable top layer comprising a plurality of apertures for removing moisture away from a wearer's skin;
(ii) a plurality of intermediate permeable layers, each having a surface area, opposed ends defining a major axis therebetween, and opposed sides defining a width therebetween;
(iii) at least one of the plurality of intermediate permeable layers comprising alternating ridges and valleys for directing moisture in a first direction across its surface area;
(iv) at least one of the plurality of intermediate permeable layers comprising alternating ridges and valleys for directing moisture in a second direction across its surface area;
(v) a substantially moisture impervious bottom layer; and
(c) wherein the moisture management pad overlies and is attached to the inner surface of the crotch section.

24. The panty construction of claim 23 wherein the permeable layers are selected from the group consisting of a layer having alternating ridges and valleys formed parallel to a major axis, a layer having alternating ridges and valleys formed substantially perpendicular to the major axis, and a layer having alternating ridges and valleys formed substantially diagonal to the major axis.

25. The panty construction of claim 23 wherein the permeable layers are formed of non-woven material.

26. The panty construction of claim 25 wherein the non-woven material is selected from the group consisting of polyester, polypropylene, nylon, cotton, and blends thereof.

27. The panty construction of claim 23 wherein the top layer further comprises a bottom surface having a plurality of spaced apart, downwardly projecting dimples.

28. The panty construction of claim 23 wherein the moisture management pad is attached to the inner surface of the crotch section by stitching or welding around the periphery of at least some of the opposed side edges and opposed front and back edges of the crotch section.

29. The panty construction of claim 22 wherein the moisture management pad is releasably attached to the inner surface of the crotch section.

30. The panty construction of claim 29 wherein the moisture management pad is releasably attached by fasteners selected from the group consisting of snaps, buttons, tapes, hook and loop material, slings, harnesses, and pockets.
